(19) United States
(12) Patent Application Publication
(10) Pub. No.: US 2009/0326591 A1
(43) Pub. Date: Dec. 31, 2009

(54) SYSTEM AND METHOD TO POSITION AND SECURE FRACTURED BONES

(76) Inventor: Edwin E. Spencer, JR., Knoxville, TN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/362,027

(22) Filed: Jan. 29, 2009

Related U.S. Application Data
(60) Provisional application No. 61/024,327, filed on Jan. 29, 2008.
(60) Provisional application No. 61/052,475, filed on May 12, 2008.

(51) Int. Cl. A61B 17/66; A61B 17/88

(52) U.S. Cl. 606/282; 606/286; 606/281

(57) ABSTRACT

A system and a method for reducing a proximal fractured humerus includes a fracture reduction plate and an elongated pin including a plurality of threads. The elongated pin is configured to be inserted through the fracture reduction plate and engage with a humeral head. The fracture reduction plate is placed on the fractured humerus and provisionally secured. Sutures may be used to guide the fracture reduction plate into place on the fractured humerus. Next, the elongated pin is engaged with the humeral head at a superior angle relative to the humerus and the fracture is reduced. The elongated pin may be used to push the humeral head in a superior direction while pulling on the humeral head using the sutures. The fracture reduction plate can then be secured to the humerus and the elongated pin removed. Bone graft material may be applied to the fracture reduction site.

### CROSS-REFERENCE TO RELATED APPLICATIONS

This application claims the benefit of U.S. provisional application Ser. No. 61/024,327, filed Jan. 29, 2008, and entitled SYSTEM AND METHOD TO POSITION AND SECURE FRACTURED BONES, and U.S. provisional application Ser. No. 61/052,475, filed May 12, 2008, and entitled SYSTEM AND METHOD TO POSITION AND SECURE FRACTURED BONES, both of which are incorporated herein in their entirety by reference.

### TECHNICAL FIELD

The present invention relates to orthopedic surgery to repair fractured bones. More particularly, the present invention relates to systems and methods for repairing proximal humeral head fractures.

### BACKGROUND

Proximal humerus fractures have been estimated conservatively to account for 5% of all fractures. These fractures occur primarily in older patients, many of whom suffer from osteoporosis. Like hip fractures, proximal humerus fractures are a major cause of morbidity in the elderly population. The most common mechanism for proximal humerus fractures is a fall on an outstretched hand from a standing height. In younger patients, high-energy trauma is a more frequent cause, and the resultant injury is more devastating. As the population base ages, the incidence of these fractures will continue to increase.

The management of proximal humerus fractures has changed with the advent of the locking plate. Rather than replacing the shoulder joint, there is now a trend to repair humeral head fractures. When compared to non-locking plates and blade plates, locking plates potentially provide better fixation which should translate into better range of motion (ROM) and increased union rates. At least one challenge associated with repairing proximal humerus fractures is the reduction of the fracture without stripping the surrounding tissue. Another challenge facing surgeons is the challenge of holding the fracture in reduction while placing the fixation plate to secure the fracture.

### BRIEF SUMMARY OF THE INVENTION

In some embodiments, the present invention is a system for positioning and securing fractured bone parts including a fracture reduction plate and an elongated pin. In certain embodiments, the fracture reduction plate includes a body portion and a head portion. The body portion includes one or more distal screw holes and an elongated hole. The head portion includes one or more proximal locking screw holes having an inner diameter. In one embodiment, the proximal locking screw hole(s) facilitate longitudinal and latitudinal movement of the elongated pin in the proximal locking screw hole. A plurality of suture holes are located around a periphery of the head portion. The elongated pin includes a pin shaft having a distal portion and a proximal portion including a plurality of threads located on the pin shaft. In one embodiment, the pin shaft of the elongated pin has an outer diameter smaller than an inner diameter of the proximal locking screw hole.

In some embodiments, the system includes a plate assembly secured to the head portion of the fracture reduction plate. The plate assembly includes a plate having a cut-out portion including a notch. In one embodiment, the plate is secured to the head portion of the fracture reduction plate such that the cut-out portion is disposed over the proximal locking screw hole. The plate assembly can also include a locking member. The locking member is engaged with the shaft of the elongated pin to secure the position of the elongated pin relative to the humeral shaft.

In still other embodiments, the present invention is a method of reducing a fracture. In some embodiments, the fracture is any one of a two-part, three-part, or four-part proximal humerus fracture.

According to one embodiment, the method can include provisionally securing a fracture reduction plate to a fractured bone and engaging an elongated pin, as described above, with the head of the bone. The fraction reduction plate includes a head and a shaft. In one embodiment, the pin is secured at a superior angle relative to the bone shaft. The fracture is then reduced. Once the fracture is reduced, the fracture reduction plate can be secured to the bone and the elongated pin removed.

According to some embodiments, the method includes securing a plurality of sutures into a muscular tissue surrounding a fractured bone and guiding a fracture reduction plate onto the fractured bone using the sutures.

In one further embodiment, the method includes simultaneously engaging the elongated pin with the head of the bone and pulling on the sutures secured to the muscular tissue to stabilize the fracture.

In another further embodiment, the method includes securing a plate assembly including a plate and a cut-out portion to the head portion of the fracture reduction plate such that the cut-out portion is disposed over the proximal locking screw hole. The elongated pin is inserted through the cut-out portion and the proximal locking screw hole and into the bone.

In further embodiments, the method including adjusting the angular position of the elongated pin relative to the bone shaft. In certain embodiments, the position of the pin can be secured with a locking member.

In still another further embodiment, the method includes applying a bone graft material.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE SEVERAL VIEWS OF THE DRAWING

FIG. 1 is a schematic view of an exemplary fracture of a humerus.

FIG. 2 is a schematic view of a fracture repair system provided in accordance with various embodiments of the present invention.

FIG. 3 is a schematic view of a fracture reduction plate provided in accordance with various embodiments of the present invention.

FIGS. 4A-4C are schematic views of screws used to secure a fraction reduction plate to a bone provided in accordance with various embodiments of the present invention.

FIG. 5 is a side view of a fracture reduction plate provided in accordance with various embodiments of the present invention.

FIG. 6 is a schematic view of an elongated pin provided in accordance with various embodiments of the present invention.

FIG. 7A is a schematic view of a plate assembly secured to a fracture reduction plate according to various embodiments of the present invention.

FIG. 7B is a schematic view of the plate assembly and fracture reduction plate in use in accordance with various embodiments of the present invention.

FIG. 7C is a top down, end view of a plate assembly engaged with an elongated pin in accordance with various embodiments of the present invention.

### DETAILED DESCRIPTION OF THE INVENTION

Proximal humerus fractures can be classified according to the Neer Classification System. The Neer Classification System includes 4 segments and also rates displacement and vascular isolation. The 4 segments are as follows: greater tuberosity (I), lesser tuberosity (II), humeral head (III), and shaft (IV). According to Neer, a fracture is displaced when there is more than 1 centimeter of displacement and 45° of angulation of any one fragment with respect to the others. Muscle pulls result in displacement. The supraspinatus and infraspinatus pull the greater tuberosity superiorly and the subscapularis pulls the lesser tuberosity medially, while the pectoralis major adducts the shaft medially.

Under the Neer Classification System, proximal humerus fractures may also be referred to as a two-part, a three-part, or a four-part fracture. Two-part fractures involve any of the four parts and include at least one fragment that is displaced. Three-part fractures include a displaced fracture of the surgical neck (shaft) in addition to either a displaced greater tuberosity or lesser tuberosity fracture. Four-part fractures include displaced fractures of the surgical neck and both tuberosities.

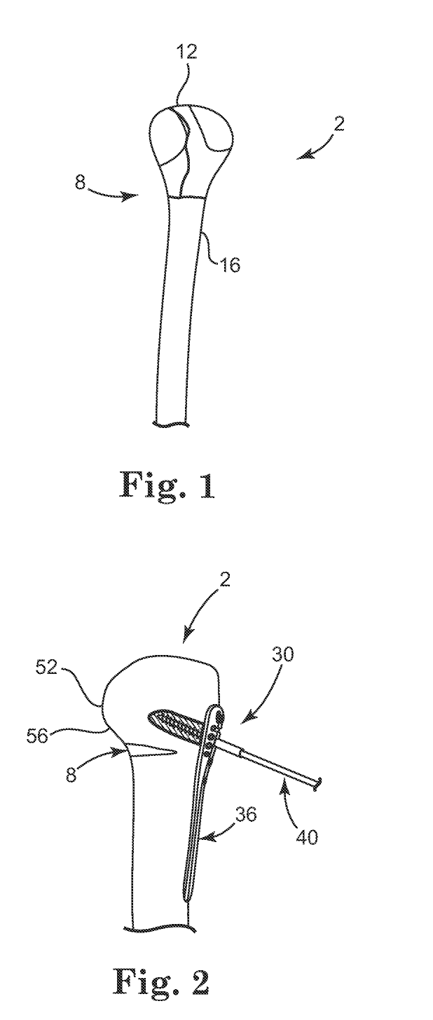

FIG. 1 is a schematic view of patient's humerus 2 including a proximal humerus fracture 8. As shown in FIG. 1, the proximal humerus fracture 8 is a two-part fracture according to the Neer Classification System. The humerus 2 is fractured at the greater tuberosity segment 12 and at the surgical neck or shaft 16. A two-part fracture is shown in FIG. 1 for simplicity of explanation. According to various embodiments of the present invention, the systems and methods described herein may be used to reduce, two-part, three-part, and four-part fractures with or without anterior or posterior displacement.

FIG. 2 is a schematic view of a fracture fixation system 30 used to repair a proximal humerus fracture 8. The fracture fixation system 30 includes a fracture reduction plate 36 and an elongated pin 40. The pin 40 is angled upwards (superiorly) into an inferior aspect 52 of the humeral head 56.

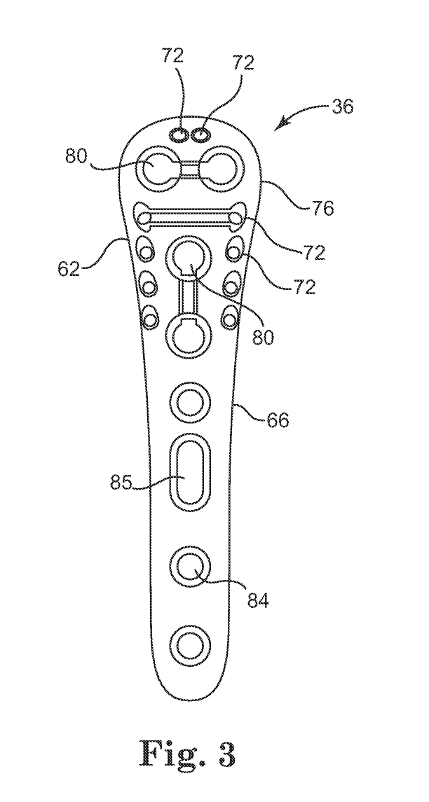

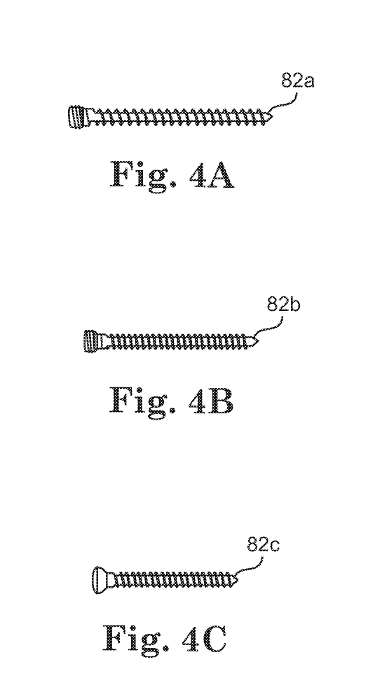

FIG. 3 is a schematic view of the fracture reduction plate 36 shown in FIG. 2 according to one embodiment of the present invention. Other possible designs for fracture reduction plates are shown in U.S. Pat. Nos. 5,190,544 (Chapman et al.); 5,954,722 (Bono); 6,623,486 (Weaver et al.); and U.S. Patent Publication No. 2007/0173839 (Running et al.) which are each hereby incorporated by reference. As shown in FIG. 3, the fracture reduction plate 36 includes a head portion 62 and a body portion 66. Two or more suture holes 72 are provided around a periphery 76 of the head portion 62 of the fracture reduction plate 36. Other suture holes may be provided at other locations on the head portion 62 and the body portion 66 of the plate. As shown in FIG. 3, the head portion 62 also includes at least one proximal locking screw hole 80. In one embodiment, the head portion 62 includes a plurality of proximal locking screw holes 80. In use, self-tapping locking screws 82a, 82b (FIGS. 4A, 4B) are inserted through the proximal locking screw holes 80 to engage the bone of the humeral head 56. The proximal locking screw holes 80 permit the screws 82a, 82b to be angled upon insertion into the bone. The screws 82a, 82b can be either cancellous self-tapping locking screws (FIG. 4A) or cortical self-tapping locking screws (FIG. 4B). In certain embodiments, lag screws may also be employed. The body portion 66 includes at least one distal compression screw hole 84. In one embodiment, the body portion 66 includes a plurality of distal compression screw holes 84. Self-tapping compression screws 82c (FIG. 4C) can be inserted into the distal compression screw holes 84 to further secure the plate 36 to the humerus 2. Additionally, the body portion 66 can include an elongated hole or slot 85. The elongated hole 85 facilitates superior and inferior translation of the fracture reduction plate 36 during initial positioning of the fracture reduction plate 36 on the humerus 2.

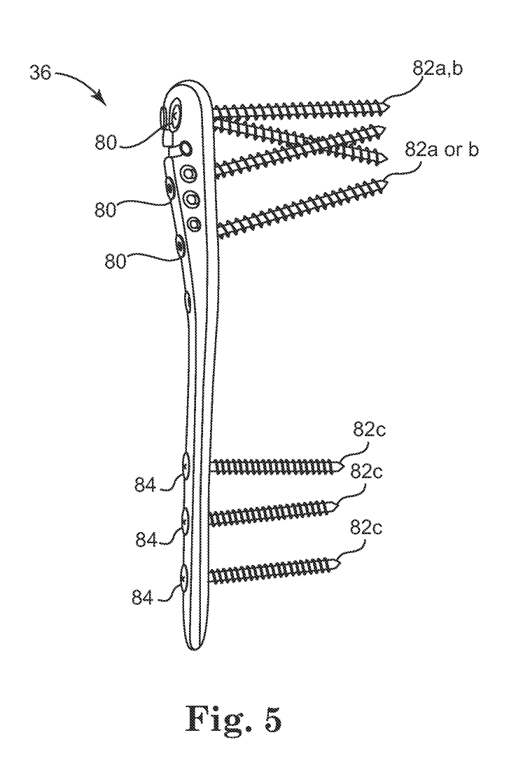

FIG. 5 is a side view of the fracture reduction plate 36 with the self-tapping locking screws 82a, 82b inserted into the proximal locking screw holes 80 and self-tapping compression screws 82c inserted into the distal compression screw holes 84. As shown in FIG. 5, the self-tapping locking screws 82a, 82b are inserted through the proximal locking screw holes 80 at an angle.

FIG. 6 is a schematic view of an elongated pin 40 provided in accordance with various embodiments of the present invention. The elongated pin 40 includes a shaft 86 extending from a proximal portion 88 to a distal portion 94. The proximal portion 88 is configured to be engaged with the humeral head 56. In one embodiment, the outer diameter of the pin shaft 86 is slightly smaller than an inner diameter of the proximal locking screw hole 80 (shown in FIG. 5) through which the elongated pin 40 is inserted during the procedure. This size differential facilitates a lateral range of motion of the elongated pin 40 in the proximal locking screw hole 80 and facilitates the elongated pin 40 to be placed in a superiorly angled position relative to the humeral shaft 16 of the humerus 2.

According to various embodiments, as shown in FIG. 6, the proximal portion 88 of the elongated pin 40 includes a plurality of threads 98. The threads 98 are configured to threadably engage the bone of the humeral head 56. The threads 98 can have either a clockwise or counter clockwise pitch. A sufficient number of threads 98 are provided on the proximal portion 88 of the shaft 86 such the fractured potions of the humerus 2 can be engaged by the elongated pin 40.

In some embodiments, the elongated pin 40 also includes a grip 102. The grip 102 is located towards the distal portion 94 of the shaft 86 and facilitates gripping of the elongated pin 40 by the surgeon performing the procedure. In one embodiment, the grip 102 may be ergonomically shaped to guide placement of the surgeon's fingers. In another embodiment, as shown in FIG. 6, the grip 102 can be substantially cylindrical. A number of grip marks 106 or other surface roughening features may be provided on the outer surface 110 of the grip 102 to facilitate a firm grasp of the elongated pin 40 by the surgeon as well as to prevent slippage of the surgeon's fingers. The surgeon grasps the grip 102 in his/her fingers and uses a rotational motion to turn the elongated pin 40 in either a clockwise or counterclockwise manner, engaging it within the bone of the humeral head 56.

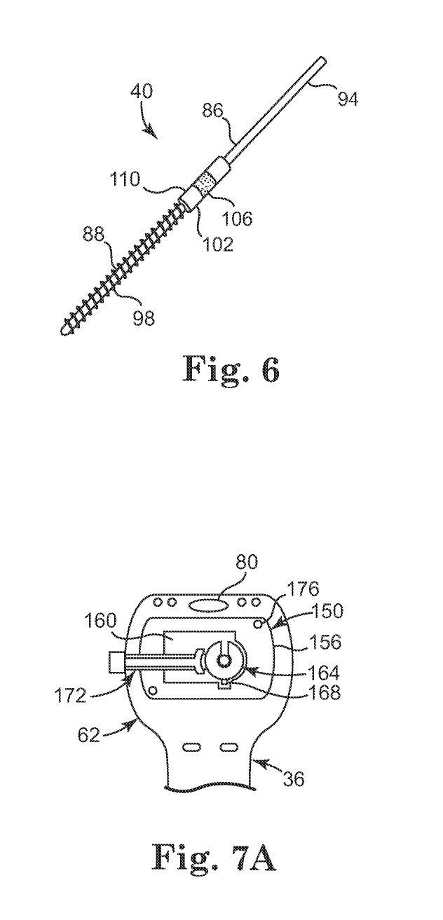

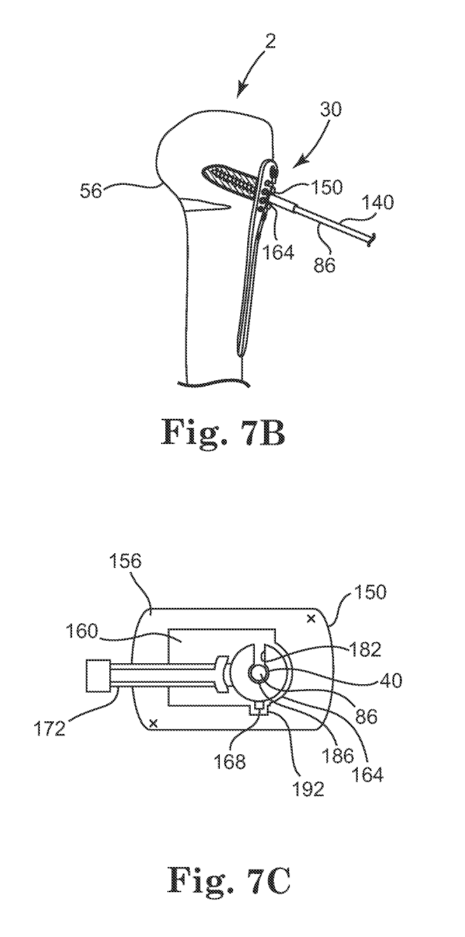

In some embodiments, the fracture fixation system 30 (shown in FIG. 2), as discussed above, also includes a plate assembly 150 that can be mounted to a fracture reduction plate 36 secured to a fractured humerus. FIG. 7A is a schematic view of a plate assembly 150 secured to a head portion 62 of a fracture reduction plate 36 according to an embodiment of the present invention. The plate assembly 150 facilitates adjustment of the position of a first bone fragment relative to a second bone fragment to reduce a fracture. As shown in FIG. 7A, the plate assembly 150 includes a plate 156 including a cut-out portion 160, a locking member 164 including a stem 168, and at least one compression screw 172. The plate 156 is adapted to be secured to a head portion 62 of a fraction reduction plate 36 using one or more screws 176 or other fastening structures. The plate 156 is positioned on the head portion 62 of the fracture reduction plate 36 such that the cut-out portion 160 is disposed over one of the proximal locking screw holes 80. In some embodiments, the cut-out portion 160 is a hole configured to be aligned with one of the proximal locking screw holes 80 located in the head portion 62 of the fracture reduction plate 36.

Once the plate 156 is secured to the fracture reduction plate 36, an elongated pin 40, such as described above, is passed through the cut-out portion 160 positioned over a selected proximal locking screw hole 80. FIG. 7B is a schematic view of the plate assembly 150 secured to a fracture reduction plate 36 positioned on a fractured humerus 2. As shown in FIG. 7B, the elongated pin 40 is passed through the plate assembly 150 and the fracture reduction plate 36 and into the bone of humeral head 56. In one embodiment, the shaft 86 of the elongated pin 40 has an outer diameter slightly smaller than an inner diameter of the proximal locking screw hole 80 in the fracture reduction plate 36 through which the elongated pin 40 is passed. This configuration facilitates a lateral range of motion of the pin 40 in the fraction reduction plate 36 and the plate assembly 150. In embodiments where the plate 156 includes a hole corresponding to a proximal locking screw hole 80, the hole in the plate 156 also has an inner diameter slightly larger than an outer diameter of the pin shaft 86.

According to one embodiment, as indicated by the directional arrows provided in FIG. 7B, the plate assembly 150 facilitates both longitudinal and latitudinal movement of the elongated pin 40 relative to the bone shaft 16. The position of the bone fragments relative to one another can be adjusted by pivoting or rotating the elongated pin 40 with respect to a longitudinal axis of the elongated pin 40 within the proximal locking screw hole 80. In one embodiment, the elongated pin 40 serves as a lag screw and pulls the position of the bone fragments into alignment with every rotation of the elongated pin 40. Once a satisfactory position of the bone fragments has been obtained, the elongated pin 40 can be locked into place relative to its longitudinal and latitudinal position using the locking member 164.

FIG. 7C is a top down, end view of the plate assembly 150 including the locking member 164 engaged with the elongated pin 40 located within a proximal locking screw hole 80. The fracture reduction plate 36 is not shown in this figure for ease of understanding. As shown in FIG. 7C, the locking member 164 includes a slot 182 having a substantially U-shaped base 186 configured to receive and engage the shaft 86 of the elongated pin 40. According to various embodiments, the cut-out portion 160 of the plate 156 includes a notch 192 configured to mate with the stem 168. Once the elongated pin 40 is properly positioned and the fracture has been reduced, the locking member 164 is placed around the shaft 86 of the elongated pin 40 and rotated until the stem 168 is mated with the notch 192 provided in the plate 156. A compressive force is then applied to the locking member 164 using the compression screw 172 or other securing structures to secure the elongated pin 40 in its final position. Once the surgical procedure is completed, the plate assembly 150 can be disassembled and the elongated pin 40 removed.

The fracture fixation system 30, as described above, according to the various embodiments of the present invention, can be used to reduce a proximal humeral fracture while holding the reduction through the fracture reduction plate 36 and potentially avoiding stripping the surround tissue. First, standard orthogonal x-rays (anteroposterior view, axillary lateral view, scapular-y or transcapular lateral views) are utilized to determine fracture pattern and appropriate indication for surgery. Other imaging techniques can also be employed to assess the nature of the fracture and indication for treatment. Next, exposure of the proximal end of the humerus 2 is facilitated through a modified beach-chair position. In one embodiment, the patient is placed in a beach-chair position and angled approximately 30-45 degrees cephalad to the floor. The arm should be prepared and draped free, and the patient should be positioned lateral enough to allow for full extension and adduction of the arm and elbow during the procedure. In addition, the posterior aspect of the shoulder should be exposed so that fluoroscopic imaging can be utilized intraoperatively to guide fracture reduction and implant positioning. In certain embodiments, a radiolucent deltoid retractor may be used to obtain an unhindered view of the fracture. Additionally, a sterile articulated arm holder (McConnell Orthopedics, Greenville, Tex.) may be used to facilitate positioning the arm during exposure, reduction, and plating.

A deltopectoral approach can be utilized to gain access to the fracture site. In some cases, a superior approach may also be used. The skin incision begins from the inferior tip of the coracoid process and extends to the deltoid insertion. While smaller incisions can be utilized in select cases, the necessity of humeral shaft exposure dictates the length of the incision. The cephalic vein is identified in the deltopectoral interval and retracted medially with the pectoralis major. All dissection is performed in the lateral aspect of the deltopectoral interval (under the deltoid and lateral to the bicipital groove), in order to avoid iatrogenic injury to the anterior humeral circumflex artery and its ascending arcuate branch that vascularizes the humeral head 56. In order to facilitate exposure, the arm is placed in abduction and internal rotation to relax the deltoid. The subdeltoid shelf is elevated and the space superior to the deltoid insertion developed, taking care not to injure the lateral branch of the axillary nerve as it enters the deltoid. In some cases, the anterior 30% of the deltoid insertion on the humeral shaft 16 can be tagged with heavy non-absorbable suture and released in a subperiosteal fashion to facilitate exposure of the posterolateral humeral head and shaft. This “distal deltoid detachment” is repaired at the end of the procedure.

The fracture fragments are exposed with minimally invasive dissection, in order to avoid further iatrogenic injury. Again, all dissection should be performed lateral to the bicipital groove to prevent injury to the proximal humeral vasculature. With three-part and select four-part fractures, a small soft tissue “window” can be created by opening the rotator interval superior to the bicipital groove. This allows for identification of the articular surface margin, anatomical reduction of the greater and lesser tuberosity fragments, and confirmation of extra articular screw position in the humeral head 56. If the rotator interval is violated in this fashion, a biceps tenodesis (usually soft tissue in the bicipital groove) is recommended to avoid adhesions of the biceps and postoperative anterior shoulder pain.

Next, the humeral fracture is reduced according to the following steps. First, mattress stitches are placed into the teres minor muscle, infraspinatus muscle, supraspinatus muscle, and the subscapularis muscle. A non-absorbable braided suture material such as, for example, No. 2 Fiber-wire, may be used for the mattress stitches. Once the stitches are secured in the surrounding tissue, the sutures are then passed through the suture holes 72 located in the fracture reduction plate 36. The sutures are used to guide the fracture reduction plate 36 down onto the humerus 2.

Next, the appropriate plate height and plate position is approximated and the fracture reduction plate 36 is provisionally secured to the shaft 16 of the humerus 2. In some embodiments, the fracture reduction plate 36 can be provisionally secured to the humeral shaft 16 using a clamp or other similar device. In other embodiments, a screw or other fastening members may be used to provisionally secure the fracture reduction plate 36 to the humeral shaft 16.

According to some embodiments, the elongated pin 40 is then inserted through the most inferior proximal locking screw hole 80 provided on the fracture reduction plate 36 and is angled up into the most inferior aspect of the humeral head 56. The surgeon pivots or rotates the elongated pin 40 to engage the bone and to stabilize and push the humeral head 56 out of varus. While the elongated pin 40 is being engaged into the bone, the sutures inserted through the suture holes are used to pull the humeral head 56 out of varus and control the humeral head 56 in the anterior-posterior plane. The elongated pin 40 is used to push the head in a superior direction and stabilize the humeral head 56 during reduction.

In other embodiments, a plate assembly 150, discussed above, is secured to the fracture reduction plate 36. An elongated pin 40 is passed through the plate assembly 150 and the fracture reduction plate 36 and inserted into the bone of humeral head 56. The plate assembly 150 facilitates both longitudinal and latitudinal movement of the elongated pin 40 relative to the longitudinal axis of the humerus 2. The position of the bone fragments relative to one another are adjusted by pivoting or rotating the elongated pin 40 with respect to a longitudinal axis of the elongated pin 40 within the proximal locking screw hole 80. In one embodiment, the elongated pin 40 acts as a lag screw and pulls the position of the bones fragments into alignment with every rotation of the elongated pin 40. Once a satisfactory position of the bone fragments has been obtained, the elongated pin 40 is locked into place relative to its longitudinal and latitudinal position using the locking member 164 and compression screw 172.

Once the fracture has been adequately reduced and secured in place using the elongated pin 40, the fracture reduction plate 36 is secured to the humeral shaft 16 and the proximal locking screws 82a, 82b are inserted through the proximal locking screw holes 80 to secure the humeral head 56. The elongated pin 40 can then be removed from the humerus. Bone graft material can be applied to the fracture reduction plate 36 if necessary or desired.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

1. A system for positioning and securing fractured bone parts, the system comprising:
a fracture reduction plate comprising:
a body portion including one or more distal screw holes and an elongated hole;
a head portion including one or more proximal locking screw holes having an inner diameter; and
a plurality of suture holes located around a periphery of the head portion; and
an elongated pin including a pin shaft having a distal portion, a proximal portion and a plurality of threads located on the shaft, wherein the proximal locking screw hole facilitates longitudinal and latitudinal movement of the elongated pin in the proximal locking screw hole.

2. The system according to claim 1, further comprising a plate assembly adapted to be secured to the head portion of the fracture reduction plate, the plate assembly including a locking member having a stem with an outer dimension, a cut-out portion including a notch generally corresponding to the outer dimension of the stem, and a compression member adapted to engage and secure the locking member.

3. The system according to claim 1, further comprising sutures adapted to be threaded through the suture holes located around the periphery of the head portion of the fracture reduction plate.

4. The system according to claim 1, wherein the pin shaft has a smaller outer diameter than the inner diameter of the proximal locking screw holes provided in the head portion of the fracture reduction plate.

5. The system according to claim 1, wherein the elongated pin further comprises a grip located on the pin shaft.

6. The system according to claim 5, wherein the grip comprises a substantially cylindrical body having an outer surface with a plurality of grip marks.

7. The system according to claim 5, wherein the grip comprises an ergonomic shape adapted to guide placement of a surgeon's fingers.

8. A system for reducing and securing a proximal fractured humerus, the system comprising:
a fracture reduction plate adapted to be secured to a patient's fractured humerus, the fracture reduction plate comprising:
a body portion including one or more distal screw holes and an elongated hole;
a head portion including at least one locking screw hole having an inner diameter; and
a plurality of suture holes located around a periphery of the head portion;
a plate secured to the head portion of the fracture reduction plate, the plate comprising a cut-out portion including a notch, wherein the plate is secured to the head portion of the fracture reduction plate such that the cut-out portion is disposed over the at least one locking screw hole; and
an elongated pin comprising a pin shaft having a distal portion and a proximal portion including a plurality of threads located on the pin shaft, the pin shaft having an outer diameter smaller than an inner diameter of the locking screw hole located in the head portion of the fracture reduction plate, wherein when the elongated pin is inserted through the locking screw hole, the locking screw hole facilitates longitudinal and latitudinal movement of the elongated pin in the locking screw hole.

9. The system according to claim 8, further comprising a locking member engaged with the pin shaft.

10. The system according to claim 9, further comprising a compression screw engaged with the locking member such that the elongated pin is secured in a desired position within the humerus.

11. A method of reducing a proximal fractured humerus, the method comprising:
securing a plurality of sutures into a muscular tissue surrounding a patient's fractured humerus, wherein the fractured humerus includes a humeral head and a humeral shaft;
guiding a fracture reduction plate onto the fractured humerus using the sutures, wherein the fracture reduction plate comprises a head portion including one or more proximal locking screw holes;
engaging an elongated pin comprising a pin shaft extending from a distal portion to a proximal portion, wherein the pin shaft includes a plurality of threads for engaging with the humeral head;
pulling on the humeral head using the sutures secured to the muscular tissue; and
reducing the proximal fractured humerus.

12. The method according to claim 11, further comprising securing the fracture reduction plate to the fractured humerus.

13. The method according to claim 11, further comprising removing the elongated pin from the fractured humerus.

14. The method according to claim 11, further comprising applying a bone graft material to the fracture reduction plate.

15. The method according to claim 11, further comprising securing the elongated pin at a superior angle relative to the humeral shaft.

16. The method according to claim 15, wherein the elongated pin is secured by engaging a locking member with the elongated pin.

17. The method according to claim 11, further comprising inserting the elongated pin through the proximal locking screw hole at a superior angle relative to the humeral shaft.

18. The method according to claim 11, wherein engaging the elongated pin with the humeral head comprises pushing the humeral head in a superior direction.

19. The method according to claim 11, further comprising inserting the elongated pin through the proximal locking screw hole and adjusting the position of the elongated pin in a latitudinal or longitudinal direction relative to the humeral shaft.

20. The method according to claim 11, further comprising simultaneously engaging the elongated pin in the humeral head and pulling the sutures to control and stabilize the humeral head.

21. The method according to claim 11, wherein engaging the elongated pin with the humeral head further comprises lagging two or more bone fragments together.

22. The method according to claim 11, wherein engaging the elongated pin with the humeral head comprises rotating the elongated pin in a clockwise or counterclockwise direction.

23. The method according to claim 11, wherein the fractured humerus is any one of a two-part, three-part, or four part fracture.

24. A method of reducing a fractured bone, the method comprising:
provisionally securing a fracture reduction plate including a head portion having one or more proximal locking screw holes onto the fractured bone, wherein the fractured bone includes a head and a shaft;
securing a plate assembly including a plate having a cut-out portion to the head portion of the fracture reduction plate, wherein the cut-out portion is disposed over the proximal locking screw hole;
engaging an elongated pin comprising a pin shaft extending from a distal portion to a proximal portion, wherein the pin shaft includes a plurality of threads for engaging with the head of the fractured bone; and
reducing the fractured bone.

25. The method according to claim 24, further comprising securing a plurality of sutures into a muscular tissue surrounding the fractured bone and guiding the fracture reduction plate onto the fractured bone using the sutures.

26. The method according to claim 25, further comprising simultaneously engaging the head of the bone with the elongated pin and pulling on the sutures to control and stabilize the fractured bone.

27. The method according to claim 24, wherein engaging the elongated pin in the head of the fractured bone further comprises lagging two or more bone fractures together.

28. The method according to claim 24, further comprising positioning the elongated pin at an superior angle relative to the shaft of the fractured bone and securing the position of the elongated pin.

29. The method according to claim 24, wherein engaging the elongated pin in the head of the fractured bone comprises inserting the elongated pin through the cut-out portion and the proximal locking screw hole.

30. The method according to claim 24, further comprising securing the fracture reduction plate to the fractured bone.

31. The method according to claim 24, further comprising removing the elongated pin and the plate assembly.

32. The method according to claim 24, wherein the fractured bone is a proximally fractured humerus.
